(19) United States
(12) Patent Application Publication
(10) Pub. No.: US 2025/0059112 A1
(43) Pub. Date: Feb. 20, 2025

(54) METHOD FOR MANUFACTURING AROMATIC HYDROCARBON, METHOD FOR MANUFACTURING POLYMER, AND APPARATUS FOR MANUFACTURING AROMATIC HYDROCARBON

(71) Applicant: Toray Industries, Inc., Chuo-ku, Tokyo (JP)

(72) Inventors: Tomoyuki ODASHIMA, Nagoya-shi, Aichi (JP); Kodai MINAMI, Nagoya-shi, Aichi (JP); Daijiro TSUKAMOTO, Nagoya-shi, Aichi (JP)

(73) Assignee: Toray Industries, Inc., Chuo-ku, Tokyo (JP)

(21) Appl. No.: 18/724,241

(22) PCT Filed: Dec. 21, 2022
(86) PCT No.: PCT/JP2022/047113

§ 371 Date: Jun. 26, 2024

(30) Foreign Application Priority Data
Dec. 27, 2021 (JP) 2021-212191

(51) Int. Cl. C07C 2/86; B01J 8/02; C07C 1/247

(52) U.S. Cl. CPC C07C 2/865; B01J 8/0278; C07C 1/247; B01J 2208/00539; C07C 2529/40

(57) ABSTRACT

The purpose of the method for manufacturing an aromatic hydrocarbon according to the present invention is to provide a manufacturing method for efficiently synthesizing a high purity aromatic hydrocarbon by a continuous reaction. To achieve the above purpose, this method for manufacturing an aromatic hydrocarbon brings ethanol and/or ethylene, and a furan derivative into contact with a catalyst in a continuous reactor.

### CROSS REFERENCE TO RELATED APPLICATIONS

This application is the U.S. National Phase of PCT/JP2022/047113, filed Dec. 21, 2022 which claims priority to Japanese Patent Application No. 2021-212191, filed Dec. 27, 2021, the disclosures of these applications being incorporated herein by reference in their entireties for all purposes.

### FIELD OF THE INVENTION

The present invention relates to a method for manufacturing an aromatic hydrocarbon, a method for manufacturing a polymer, and an apparatus for manufacturing an aromatic hydrocarbon.

### BACKGROUND OF THE INVENTION

In recent years, efforts to achieve carbon neutral have been accelerated worldwide due to concerns about global warming caused by greenhouse gases including carbon dioxide. Accordingly, in the material field, non-petroleum-derived materials represented by polylactic acid and the like have been actively developed and adopted.

Polyethylene terephthalate (PET) widely used in applications such as fibers and films is also originally a petroleum-derived material. In recent years, replacement of PET with non-petroleum-derived materials, particularly biomass-derived materials, has been studied. Specifically, among monomers used for the manufacture of PET, biomass-derived material compounds of monoethylene glycol have already been put on the market, and the monomers have been partly adopted. On the other hand, as for terephthalic acid, which is another monomer, biomass-derived material compounds are strongly demanded, and development is actively conducted.

Currently, as terephthalic acid used industrially, mainly petroleum-derived para-xylene is a starting material of the synthesis. Therefore, methods for manufacturing para-xylene from a biomass raw material have been studied. Among them, a method has been proposed in which 2,5-dimethylfuran, which can be synthesized from a sugar chain compound such as glucose and fructose, is used and converted into para-xylene in one step by a reaction with ethylene or ethanol in the presence of a catalyst (for example, Patent Documents 1 to 3).

In addition, studies on conversion into ethylene by a dehydration reaction using ethanol obtained by fermentation are also actively conducted, and these are also based on biomass raw materials.

### PATENT DOCUMENTS

Patent Document 1: WO 2009/110402
Patent Document 2: WO 2013/040514
Patent Document 3: Japanese Patent Laid-open Publication No. 2017-137293

### SUMMARY OF THE INVENTION

All the methods for manufacturing para-xylene in the prior art are based on batch reactions, and continuous reactions, which are industrially advantageous, are demanded. In addition, side reactions such as hydrolysis of 2,5-dimethylfuran as a raw material with by-product water and isomerization and disproportionation of generated para-xylene may proceed, and thus radical suppression thereof is desired.

An object of the present invention is to provide a method for manufacturing an aromatic hydrocarbon, in which a high-purity aromatic hydrocarbon is efficiently synthesized by a continuous reaction.

The present invention adopts the following means in order to solve the problems. That is, the present invention relates to the following.
[1] A method for manufacturing an aromatic hydrocarbon, including contacting ethanol and/or ethylene, and a furan derivative with a catalyst in a continuous reactor.
[2] The method for manufacturing an aromatic hydrocarbon according to [1], in which ethanol is brought into contact with the catalyst in a continuous reactor to convert at least a part of the ethanol into ethylene, and the ethylene and the furan derivative are brought into contact with the catalyst in a continuous reactor.
[3] The method for manufacturing an aromatic hydrocarbon according to [2], in which the conversion of ethanol to ethylene and the contact of ethylene and the furan derivative with the catalyst are performed in the same continuous reactor.
[4] The method for manufacturing an aromatic hydrocarbon according to any one of [1] to [3], in which ethanol and/or ethylene and the furan derivative are brought into contact with the catalyst in a gaseous state.
[5] The method for manufacturing an aromatic hydrocarbon according to any one of [1] to [4], in which a molar ratio of ethanol and/or ethylene (in a case where both ethanol and ethylene are contained, a total of ethanol and ethylene) to the furan derivative to be brought into contact with the catalyst is 1.0 or more and 50.0 or less.
[6] The method for manufacturing an aromatic hydrocarbon according to any one of [1] to [5], in which a pressure in the continuous reactor is 1.0 MPa or less.
[7] The method for manufacturing an aromatic hydrocarbon according to any one of [1] to [6], in which the at least one catalyst contains a solid acid.
[8] The method for manufacturing an aromatic hydrocarbon according to [7], in which the solid acid is at least one selected from the group consisting of zeolite, alumina, and a heteropolyacid.
[9] The method for manufacturing an aromatic hydrocarbon according to any one of [1] to [8], in which the furan derivative is derived from biomass.
[10] The method for manufacturing an aromatic hydrocarbon according to any one of [1] to [9], in which ethanol and/or ethylene is derived from biomass.
[11] An aromatic hydrocarbon obtained by the method for manufacturing an aromatic hydrocarbon according to any one of [1] to [10].
[12] A method for manufacturing a polymer, including: manufacturing the aromatic hydrocarbon by the method for manufacturing an aromatic hydrocarbon according to any one of [1] to [10]; and manufacturing a polymer using the resulting aromatic hydrocarbon as a raw material.
[13] An apparatus for manufacturing an aromatic hydrocarbon, including: a raw material supply part; a flow type continuous reactor filled with a catalyst; and a reaction mixture recovery part, in which the raw material supply part includes supply means for continuously supplying a raw material compound containing ethanol and/or ethylene, and a furan derivative to the continuous reactor, and the reaction mixture recovery part includes discharge means for continuously extracting a reaction mixture having been in contact with the catalyst from the continuous reactor.
[14] The apparatus for manufacturing an aromatic hydrocarbon according to [13], in which the raw material supply part further includes vaporization means for vaporizing ethanol and the furan derivative.
[15] The apparatus for manufacturing an aromatic hydrocarbon according to or [14], in which the reaction mixture recovery part further includes condensation means for condensing at least a part of the extracted reaction mixture.
[16] The apparatus for manufacturing an aromatic hydrocarbon according to any one of [13] to [15], further including pressure control means capable of controlling an internal pressure of the continuous reactor within a range of 1.0 MPa or less.

According to the present invention, it is possible to provide a method for manufacturing an aromatic hydrocarbon by an industrially advantageous continuous reaction in which a side reaction is suppressed.

### BRIEF DESCRIPTION OF THE DRAWINGS

FIG. 1 schematically shows a configuration of a manufacturing apparatus used in examples of the present invention.

### DETAILED DESCRIPTION OF EMBODIMENTS OF THE INVENTION

Preferable embodiments of the present invention will be described below in detail. The present invention is not limited only to the embodiments described below but should be understood to also include various modifications implemented without changing the gist of the present invention.

### (1) Raw Material Compound

In a method for manufacturing an aromatic hydrocarbon of the present invention, raw material compounds are ethanol and/or ethylene, and a furan derivative. Each raw material compound may be a commercially available product, a synthetic product by a known technique, or a synthetic product by a new method. For each raw material compound, either a petroleum-derived material compound or a biomass-derived material compound can be used in the same manner.

Among them, in the method for manufacturing an aromatic hydrocarbon of the present invention, the furan derivative is preferably derived from biomass. In the method for manufacturing an aromatic hydrocarbon of the present invention, ethanol and/or ethylene are preferably derived from biomass. When any of these raw material compounds or a plurality of raw material compounds is derived from biomass, the obtained aromatic hydrocarbon can be treated as an at least partially biomass-derived material compound. In particular, when all the raw material compounds are derived from biomass, the resulting aromatic hydrocarbon can also be treated as a complete biomass-derived material compound, which is most preferable. In addition, it is also preferable that ethanol, ethylene, and the furan derivative, which are raw material compounds, are recovered as unreacted components from the reaction mixture in the method for manufacturing an aromatic hydrocarbon of the present invention, separated and purified as necessary, and reused.

In the present invention, the furan derivative can be represented by following General Formula (I).

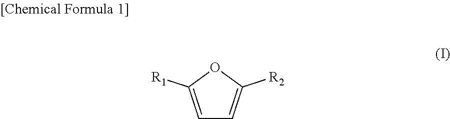

R¹ and R² in General Formula (I) are each a substituent selected from hydrogen, an alkyl group having one to six carbon atoms, and an alkyl group having one to six carbon atoms having a substituent, and R¹ and R² may be the same as or different from each other.

The furan derivative is preferably a 2,5-dialkylfuran, particularly preferably 2,5-dimethylfuran.

Ethanol and ethylene can be used singly or as a mixture. When they are used as a mixture, a predetermined amount may be weighed and used in consideration of the total number of moles of both components. The composition of both components when used as a mixture is not particularly limited.

In the present invention, a mixture containing ethylene converted from ethanol by a dehydration reaction and ethanol that has not reacted at that time can also be used as ethylene.

### (2) Reaction

In the method for manufacturing an aromatic hydrocarbon of the present invention, ethanol and/or ethylene and the furan derivative are brought into contact with a catalyst in a continuous reactor. Here, the reaction that preferably proceeds is as in following Formula (1). When ethanol is contained in the raw material compounds, first, a dehydration reaction of ethanol proceeds under the action of the catalyst described later. The dehydration reaction of ethanol converts ethanol into ethylene. Subsequently, a Diels-Alder reaction (hereinafter abbreviated as a DA reaction) of ethylene generated by the dehydration reaction of ethanol and/or ethylene used as a raw material compound with the furan derivative proceeds. The DA reaction yields a bicyclo intermediate. Further, the dehydration reaction of the bicyclo intermediate proceeds, whereby an aromatic hydrocarbon can be obtained. When the raw material compounds do not contain ethanol, there is no dehydration reaction of ethanol in following Formula (1), and the reaction starts from ethylene.

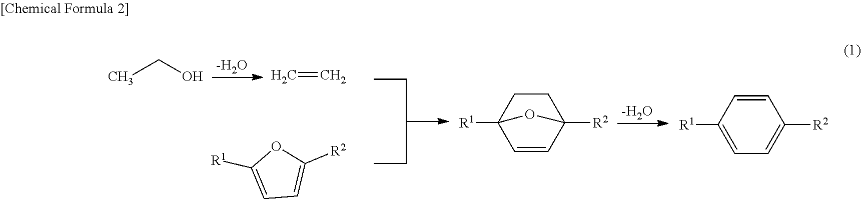

In the method for manufacturing an aromatic hydrocarbon of the present invention, it is preferable that ethanol be brought into contact with the catalyst in a continuous reactor to convert at least a part of the ethanol into ethylene, and the ethylene and the furan derivative be brought into contact with the catalyst in a continuous reactor. In the present invention, ethanol and/or ethylene is preferably derived from biomass as described above, but ethanol is more excellent in availability of biomass-derived material compounds than ethylene in the current market. Therefore, by using ethanol as a raw material and converting the raw material into ethylene, the aromatic hydrocarbon as a biomass-derived material compound can be easily obtained.

The aromatic hydrocarbon preferably obtained in the present invention is a benzene derivative represented by following General Formula (II).

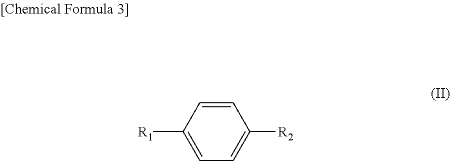

R¹ and R² in General Formula (II) are each a substituent selected from hydrogen, an alkyl group having one to six carbon atoms, and an alkyl group having one to six carbon atoms having a substituent, and R¹ and R² may be the same as or different from each other.

The aromatic hydrocarbon is more preferably a para-dialkylbenzene, particularly preferably para-xylene.

In the reaction of Formula (1) above, the catalyst described later acts on the DA reaction and the dehydration reaction. In these reactions, depending on reaction conditions, isomerization, disproportionation, and the like of the produced aromatic hydrocarbon proceed, and for example, aromatic hydrocarbons variously substituted with alkyl groups such as trialkylbenzenes, ortho-dialkylbenzenes such as ortho-xylene, meta-dialkylbenzenes such as meta-xylene, and monoalkylbenzenes such as toluene and ethylbenzene may be produced. These various aromatic hydrocarbons are preferably converted into other compounds or recovered by, for example, isomerization or adsorptive separation using a known zeolite technique. In particular, it is preferable to obtain para-xylene by such a technique.

In addition, it is known that in the reaction of Formula (1), regarding the furan derivative, generation of a by-product ring opened compound (2,5-hexanedione) associated with hydrolysis is observed. It is known that the content of the ring opened compound to the number of moles of the furan derivative as the raw material compound is at least 2 to 3% or more, and when the content is high, it is as large as 30% or more (see, for example, Non-Patent Document Angew. Chem. Int. Ed. 2016, 55, 13061-13066).

The amount of these by-products is preferably as small as possible for the main product, but in the method for manufacturing an aromatic hydrocarbon of the present invention, it has been found that the amount of by-product 2,5-hexanedione is significantly smaller than that in the known technique, and thus the present invention has been completed. That is, the by-product amount of 2,5-hexanedione in the method for manufacturing an aromatic hydrocarbon of the present invention tends to be at most 1.2% or less to the number of moles of the furan derivative as the raw material compound and is preferably 1.0% or less, more preferably 0.9% or less, still more preferably 0.8% or less. In the continuous reaction in the present invention, the by-product amount of 2,5-hexanedione is calculated as the by-product amount of 2,5-hexanedione obtained per unit time to the number of moles of the furan derivative supplied per unit time. Although the mechanism by which such a result is obtained is not clear, it is presumed that in the manufacturing method of the present invention, the conversion rate of the furan derivative in the DA reaction is extremely high, and the by-product water does not accumulate in the reaction system unlike the batch reaction of the prior art, which is advantageous for suppressing the hydrolysis of the furan derivative.

The aromatic hydrocarbon of the present invention is obtained by the method for manufacturing an aromatic hydrocarbon of the present invention.

### (3) Catalyst

In the present invention, the catalyst can include a catalyst that acts on the dehydration reaction of ethanol and a catalyst for promoting the DA reaction and/or the dehydration reaction of the bicyclo intermediate. These catalysts may be the same as or different from each other. In addition, one or a combination of two or more of catalysts having various compositions, catalyst strengths, and catalyst amounts can be selected and used.

The catalyst in the present invention is preferably an acid catalyst. Among such catalysts, in the method for manufacturing an aromatic hydrocarbon of the present invention, it is more preferable that at least one catalyst contain a solid acid. The solid acid may be one on which a metal ion or the like is supported.

In the method for manufacturing an aromatic hydrocarbon of the present invention, the solid acid is more preferably at least one selected from the group consisting of zeolite, alumina, and a heteropolyacid.

Examples of the zeolite include the MFI type (such as ZSM-5), the Y type, the beta type, and the mordenite type. Among them, the MFI type is preferable, and ZSM-5 is particularly preferable. In the zeolite, the molar ratio of SiO₂/Al₂O₃ is preferably 2 to 2,000, more preferably 3 to 200, still more preferably 4 to 100, still more preferably 5 to 50. The zeolite can contain a binder such as alumina and clay in an arbitrary ratio. The zeolite may be molded into a bead shape, a pellet shape, or the like.

Examples of the alumina include γ-alumina and η-alumina.

Examples of the heteropolyacid include phosphotungstic acid and silicotungstic acid. The heteropolyacid may be supported on a support such as silica gel in an arbitrary ratio.

### (4) Manufacturing Apparatus

An apparatus for manufacturing an aromatic hydrocarbon of the present invention includes a raw material supply part, a flow type continuous reactor filled with a catalyst, and a reaction mixture recovery part, in which the raw material supply part includes supply means for continuously supplying a raw material compound containing ethanol and/or ethylene, and a furan derivative to the continuous reactor, and the reaction mixture recovery part includes discharge means for continuously extracting a reaction mixture having been in contact with the catalyst from the continuous reactor. That is, the apparatus for manufacturing an aromatic hydrocarbon of the present invention includes the raw material supply part, the continuous reactor, and the reaction mixture recovery part as minimum constituent units. Furthermore, an apparatus used for pretreating the raw material compound, an apparatus used for separating or purifying the reaction mixture, or the like may be attached.

As described above, the apparatus for manufacturing an aromatic hydrocarbon of the present invention includes the raw material supply part including the supply means for continuously supplying the raw material compound containing ethanol and/or ethylene and the furan derivative to the continuous reactor. The supply means refers to means for transferring each raw material compound through piping using mechanical energy, pressure of gas, or the like. Specifically, use of various known pumps and high pressure gases such as nitrogen and helium can be exemplified, and preferable means may be selected according to the characteristics and state of the raw material, or a plurality of means may be combined.

In the apparatus for manufacturing an aromatic hydrocarbon of the present invention, the raw material supply part preferably further includes vaporization means for vaporizing ethanol and the furan derivative. Specific examples of the vaporization means include an apparatus that directly heats and vaporizes a liquid raw material compound, and an apparatus that prepares and supplies an air-fuel mixture with a raw material compound by passing an inert gas such as nitrogen and helium therethrough.

In the method for manufacturing an aromatic hydrocarbon of the present invention, ethanol and/or ethylene and the furan derivative are preferably brought into contact with the catalyst in a gaseous state. By bringing all the raw material compounds into contact with the catalyst in a gaseous state, the reaction result can be easily stabilized against fluctuations in temperature and pressure.

In the apparatus for manufacturing an aromatic hydrocarbon of the present invention, the raw material supply part preferably further includes raw material supply control means. The raw material supply control means refers to means for adjusting the composition and supply amount of the raw material compound. Examples of the aspect of raw material supply control include an aspect in which a certain amount of the raw material is intermittently supplied, an aspect in which the raw material is continuously supplied at a certain rate, and an aspect in which raw material supply is adjusted while monitoring a reaction result. In addition, the apparatus used as the raw material supply control means is a raw material supply control apparatus that adjusts the furan derivative and ethanol and/or ethylene, which are raw material compounds, to an arbitrary amount and supplies the resultant to the subsequent continuous reactor. The raw material supply control apparatus is not limited in structure and configuration as long as it has the above-described functions, and specifically, a container provided with a metering pump can be exemplified. In addition, the raw material supply control apparatus may be configured to supply each raw material compound individually or may be configured to supply the raw material compounds after mixing the raw material compounds.

As described above, the apparatus for manufacturing an aromatic hydrocarbon of the present invention includes a flow type continuous reactor filled with the catalyst. In the present invention, the continuous reactor refers to a flow type reactor with which supply of a raw material compound and discharge of a reaction mixture can be simultaneously performed. In the present invention, the continuous reactor is distinguished from a sealed vessel or a batch reactor in which charged raw material compounds and a reaction mixture are substantially confined in the system by reflux. That is, in the present invention, it is sufficient that the raw material compounds flow inside the continuous reactor, and after the contact with the catalyst, the generated reaction mixture is discharged without being confined in the continuous reactor.

The continuous reactor in the present invention can also be constantly aerated with an inert gas that is not directly involved in the reaction, such as nitrogen, helium, and argon, during the reaction or before and after the reaction.

When an inert gas is used in the apparatus for manufacturing an aromatic hydrocarbon of the present invention, it is preferable to include gas flow rate control means for adjusting the flow rate of the inert gas. By adjusting the flow rate of the inert gas, the ratio of the inert gas to the raw material compound can be controlled to an arbitrary value, and a stable reaction can be performed. The gas flow rate control means may be provided in the raw material supply part or in the continuous reactor.

In the continuous reactor used in the present invention, ethanol and/or ethylene and the furan derivative supplied from the raw material supply part are brought into contact with the catalyst. Here, as described in the (2) Reaction section, conversion from ethanol to ethylene and/or conversion to the aromatic hydrocarbon by bringing the ethylene into contact with the furan derivative is performed.

When ethanol is used as a raw material, the conversion of ethanol to ethylene and the reaction of bringing the obtained ethylene and the furan derivative into contact with each other can be performed in different continuous reactors or in the same continuous reactor. When the reactions are performed in different continuous reactors, there is an advantage that an optimum catalyst and reaction conditions can be adopted for each reaction. When the reactions are performed in different continuous reactors, a plurality of raw material supply parts and a plurality of continuous reactors for respectively performing the conversion from ethanol to ethylene and the reaction of bringing the obtained ethylene into contact with the furan derivative are prepared and connected.

In the method for manufacturing an aromatic hydrocarbon of the present invention, the conversion of ethanol to ethylene and the contact of ethylene and the furan derivative with the catalyst are preferably performed in the same continuous reactor. By performing the conversion of ethanol to ethylene and the contact of ethylene and the furan derivative with the catalyst in the same continuous reactor, the manufacturing apparatus can be easily simplified.

The continuous reactor can be filled with a required amount of the catalyst described in the (3) Catalyst section, and the catalyst is preferably brought into contact with ethanol and/or ethylene and the furan derivative supplied from the previous stage to accelerate the reactions. The shape of the continuous reactor is not particularly limited, but a cylindrical tubular shape can be exemplified. The material and structure of the continuous reactor are designed so that the continuous reactor can be heated as necessary for the reaction and can withstand the pressure that can be generated by the heating.

The outlet of the continuous reactor may be branched and respectively connected to the subsequent reaction mixture recovery part and the inlet of the continuous reactor. In this case, the reaction mixture containing the unreacted raw material compound can be circulated to the continuous reactor at an arbitrary ratio.

As described above, the apparatus for manufacturing an aromatic hydrocarbon of the present invention includes the reaction mixture recovery part including the discharge means for continuously extracting the reaction mixture having been in contact with the catalyst from the continuous reactor. The discharge means refers to means for continuously extracting the reaction mixture having been in contact with the catalyst from the reaction apparatus. The apparatus used as the discharge means is not limited in structure or configuration as long as it has a function of recovering the reaction mixture generated in the preceding continuous reactor. In a preferable aspect of the present invention, since the continuous reactor is at atmospheric pressure or more, an apparatus using self-generative pressure or an apparatus using a pump can be exemplified as an apparatus used as the discharge means. In addition, these may be connected to a storage tank of the reaction mixture.

In the apparatus for manufacturing an aromatic hydrocarbon of the present invention, the reaction mixture recovery part preferably further includes condensation means for condensing at least a part of the extracted reaction mixture. The condensation means refers to means for liquefying a gaseous reaction mixture. As an apparatus used as the condensation means, a condensation apparatus having a function of cooling a high-temperature and high-pressure reaction mixture that can be generated in the preceding continuous reactor and recovering the reaction mixture at normal pressure is preferable.

The apparatus for manufacturing an aromatic hydrocarbon of the present invention preferably further includes a separation apparatus and/or a purification apparatus. By including the separation apparatus, ethanol, ethylene, and the furan derivative, which are unreacted raw material compounds, can be recovered from the reaction mixture and reused as raw materials. In addition, by including the purification apparatus, a desired aromatic hydrocarbon component can be obtained with high purity from the reaction mixture. These apparatuses may be a general product having a known mechanism or a dedicated design product.

### (5) Reaction Conditions

In the method for manufacturing an aromatic hydrocarbon of the present invention, reaction conditions such as the ratio of the raw material compounds, the amount of the raw material compounds supplied to the continuous reactor, and the reaction temperature are appropriately adjusted according to the type and the filling amount of the catalyst.

In the method for manufacturing an aromatic hydrocarbon of the present invention, the molar ratio of ethanol and/or ethylene (in the case where both ethanol and ethylene are contained, the total thereof) to the furan derivative to be brought into contact with the catalyst is not particularly limited as long as every raw material compound is contained but is preferably 1.0 or more and 50.0 or less.

The lower limit of the molar ratio is more preferably 2.0 or more, still more preferably 3.0 or more, particularly preferably 5.0 or more. In the method for manufacturing an aromatic hydrocarbon of the present invention, as the amount of ethanol and/or ethylene increases, that is, as the value of the molar ratio increases, the yield of the total aromatic hydrocarbons and the yield of para-xylene contained tend to be improved, which is advantageous. On the other hand, in the known technique (see, for example, Non-Patent Document Angew. Chem. Int. Ed. 2016, 55, 13061-13066), it has been reported that the molar ratio of ethanol to the furan derivative is most excellent at equimolar (1.0 in the present invention), and the side reaction also increases as the amount of ethanol increases. The reason why there is a difference in suitable molar ratio between the known technique and the present invention is not clear, but it is presumed that this is due to a difference between a batch reaction system in which the reaction gradually proceeds in a closed system and a continuous reaction system in which the reaction needs to proceed rapidly on a catalyst.

The upper limit of the molar ratio is more preferably 40.0 or less, still more preferably 35.0 or less, particularly preferably 20.0 or less. In the method for manufacturing an aromatic hydrocarbon of the present invention, as the amount of ethanol and/or ethylene is smaller, the proportion of ethanol and/or ethylene recovered in an unreacted state is smaller, and the manufacture of the aromatic hydrocarbon becomes more efficient.

The reaction temperature in the present invention is preferably 200° C. or more, more preferably 230° C. or more, still more preferably 250° C. or more, particularly preferably 280° C. or more, most preferably 300° C. or more. As the reaction temperature is higher, raw material consumption in the same catalyst amount tends to be promoted. On the other hand, the upper limit of the reaction temperature is not particularly limited but is about 500° C. and preferably 400° C. in consideration of reaction selectivity.

In the method for manufacturing an aromatic hydrocarbon of the present invention, the pressure in the continuous reactor is not limited, but the pressure in the continuous reactor is preferably 1.0 MPa or less, more preferably 0.5 MPa or less. The lower limit of the pressure in the continuous reactor is not particularly limited but is usually about 0.01 MPa. In the method specifically disclosed in the prior technique of a similar reaction, the internal pressure is estimated to be about 2 MPa at the lowest in a batch reaction in a sealed container. On the other hand, the reaction in the method for manufacturing an aromatic hydrocarbon of the present invention can be carried out at an internal pressure similar to that in the prior technique but can be reduced in pressure, which is advantageous from the viewpoint of installation cost of manufacturing equipment.

The apparatus for manufacturing an aromatic hydrocarbon of the present invention preferably includes pressure control means capable of controlling the internal pressure of the continuous reactor within the range of 1.0 MPa or less. By including such pressure control means, the internal pressure of the continuous reactor can be controlled within the above range. Examples of the pressure control means include control on the upstream side of the manufacturing apparatus, such as adjustment of a raw material supply amount by the raw material supply part and adjustment of the pressure of an inert gas in the case of using the inert gas, or adjustment of an extraction amount by the reaction mixture recovery part on the downstream side of the manufacturing apparatus.

### (6) Separation and Purification

The reaction mixture containing the aromatic hydrocarbon obtained by the method for manufacturing an aromatic hydrocarbon of the present invention can be separated and purified by a known method depending on the content of the aromatic hydrocarbon and the type of impurities. The obtained aromatic hydrocarbon can be used as an industrial raw material or a fuel component.

In the method for manufacturing an aromatic hydrocarbon of the present invention, when unreacted raw material compounds such as ethanol, ethylene, and the furan derivative are contained in the reaction mixture, as described above, the aromatic hydrocarbon and the raw material compounds thereof are preferably separated and recovered and further purified as necessary to be reused as a raw material compound of the aromatic hydrocarbon.

### (7) Method for Manufacturing Polymer

A method for manufacturing a polymer of the present invention includes manufacturing the aromatic hydrocarbon by the method for manufacturing an aromatic hydrocarbon of the present invention and manufacturing a polymer using the resulting aromatic hydrocarbon as a raw material. A preferable example of the method for manufacturing a polymer of the present invention is as follows. That is, first, para-xylene is manufactured by the method for manufacturing an aromatic hydrocarbon of the present invention. Next, the obtained para-xylene is converted into terephthalic acid by oxidation. Then, polyethylene terephthalate (PET) is manufactured using terephthalic acid.

### EXAMPLES

Hereinafter, the present invention will be described in detail with reference to examples. However, the present invention is not limited by these examples.

### [Manufacturing Apparatus]

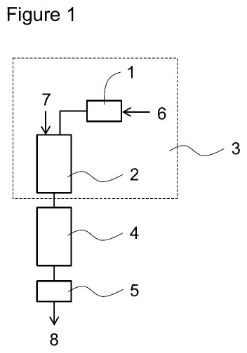

FIG. 1 shows an outline of a configuration and a function of a manufacturing apparatus used in the following examples.

### (1) Raw Material Supply Part

As a raw material supply part 3, a manufacturing apparatus including a gas flow rate controller 1 and a raw material vaporizer 2 was used. To the upper end of the raw material vaporizer 2 formed of a stainless steel tube provided with a heater, a pipe of an inert gas 6 through an airtight raw material supply port and the gas flow rate controller 1 was connected. The lower end of the raw material vaporizer 2 was connected to a continuous reactor 4 described later via a heat retaining pipe. From the raw material supply port, a raw material compound 7 was injected through a microsyringe or a micro-feeder. Here, passing of the inert gas from the gas flow rate regulator 1 and injection of the raw material compound with a microsyringe or a micro-feeder correspond to the supply means of the raw material compound.

### (2) Continuous Reactor 4

The continuous reactor 4 was a stainless steel tube equipped with a heater, into which a catalyst tube filled with the catalyst was inserted. The catalyst tube was a quartz tube having an inner diameter of 3 mm. In the quartz tube, the catalyst sandwiched between quartz wool at both ends was filled. The upper end of the continuous reactor 4 was connected to the raw material supply part, and the lower end was connected to a reaction mixture recovery part 5 by piping.

### (3) Reaction Mixture Recovery Part 5

In the reaction mixture recovery part 5, the pipe from the lower end of the continuous reactor 4 was cooled with liquid nitrogen, and a reaction mixture 8 was condensed and recovered. Here, the pressure in the continuous reactor was used as the discharge means for extracting the reaction mixture 8 from the continuous reactor 4.

### [Analysis of Reaction Mixture]

A sample from the reaction mixture recovery part was analyzed by gas chromatography (GC), each component was quantified, and the yield of each component was calculated as follows. Each component was assigned using gas chromatography-mass spectrometry (GC/MS) or an authentic sample.

$$Yield of ethylene ( in the case of ethanol raw material ) = [ amount of ethylene in reaction mixture \text{(mol)]/[amount} of ethanol supplied as raw material ( mol ) ] \times 100 ( \% )$$

$$Yield of each component of aromatic hydrocarbon or 2 , 5 - hexanedione = [ amount of component in reaction mixture \text{(mol)]/[amount} of furan derivative supplied as raw material ( mol ) ] \times 100 ( \% )$$

### Example 1

A Y type zeolite catalyst (HSZ/320HOD1C manufactured by Tosoh Corporation) was pulverized in a mortar and sieved to obtain a 40 to 60 mesh size powder, and 12 mg of the powder was filled in a catalyst tube. Subsequently, while helium was supplied at 14 mL/min in the manufacturing apparatus, the raw material supply part and the continuous reactor were heated at 200° C. and 500° C., respectively, for 1 hour, and then the temperature of the continuous reactor was lowered to 300° C. and stabilized while the raw material supply part was kept at 200° C. The reaction mixture recovery part was cooled with liquid nitrogen. Subsequently, 1 μL of an equimolar mixed solution of ethanol and 2,5-dimethylfuran was injected from the raw material supply port with a microsyringe. The reaction mixture obtained in the reaction mixture recovery part 30 minutes after injection was analyzed by GC, and the yield of each component was calculated to obtain the results shown in Table 1.

### Example 2

The same procedures as in Example 1 were carried out except that ZSM-5 (HSZ/840HOD1A manufactured by Tosoh Corporation) was used as the catalyst.

### Example 3

The same procedures as in Example 1 were carried out except that ZSM-5 (HSZ/840HOD1A manufactured by Tosoh Corporation) was used as the catalyst.

### Example 4

The same procedures as in Example 1 were carried out except that mordenite type zeolite (HSZ/690HOD1A manufactured by Tosoh Corporation) was used as the catalyst.

### Example 5

The same procedures as in Example 1 were carried out except that beta type zeolite (HSZ/940HOD1A manufactured by Tosoh Corporation) was used as the catalyst.

### Example 6

An aqueous solution of silicotungstic acid was mixed with neutral silica gel (silica gel 60N manufactured by Kanto Chemical Co., Inc.) so that the amount of silicotungstic acid was 42 wt % to the silica gel, water was distilled off, and the mixture was heated and dried to obtain a powder. The same procedures as in Example 1 were carried out except that the catalyst was changed to this powder.

### Example 7

The same procedures as in Example 6 were carried out except that silicotungstic acid was changed to phosphotungstic acid.

From the results of Examples 1 to 7 shown in Table 1, it was found that various catalysts can be applied in the method of the present invention, and ethylene production and aromatic hydrocarbons containing para-xylene are obtained.

TABLE 1-1

| | Example 1 | Example 2 | Example 3 | Example 4 | Example 5 |
|---|---|---|---|---|---|
| Catalyst species | Y type | ZSM-5 | ZSM-5 | Mordenite type | Beta type |
| SiO₂/Al₂O₃ ratio (mol/mol) | 6 | 24 | 39 | 230 | 41 |
| Ethylene (%/EtOH) | 30.8 | 34.2 | 22.5 | 23.0 | 10.4 |
| Total aromatic | 12.3 | 20.6 | 37.6 | 10.2 | 19.0 |
| hydrocarbons (%/DMF) | | | | | |
| Total xylenes (%/DMF) | 2.3 | 9.5 | 17.0 | 1.3 | 4.9 |
| PX (%/DMF) | 0.6 | 9.1 | 5.0 | 0.4 | 1.0 |
| 2,5-Hexanedione (%/DMF) | 0.1 | 0.3 | 0.8 | 0.6 | 0.1 |

TABLE 1-2

| | Example 6 | Example 7 |
|---|---|---|
| Catalyst species | Silicotungstic | Phosphotungstic |
| | acid | acid |
| SiO₂/Al₂O₃ ratio (mol/mol) | — | — |
| Ethylene (%/EtOH) | 65.4 | 41.5 |
| Total aromatic | 12.5 | 5.1 |
| hydrocarbons (%/DMF) | | |
| Total xylenes (%/DMF) | 5.3 | 3.6 |
| PX (%/DMF) | 4.5 | 3.4 |
| 2,5-Hexanedione (%/DMF) | 0.5 | 0.3 |

In Table 1, EtOH represents ethanol. DMF represents 2,5-dimethylfuran. The total xylenes represents the total xylene components including para-xylene, meta-xylene, and ortho-xylene among the total aromatic hydrocarbons. PX represents para-xylene. That is, PX represents only the para-xylene component in the total xylenes. The same applies to other tables.

### Examples 8 to 12

ZSM-5 (HSZ/840HOD1A manufactured by Tosoh Corporation) was pulverized in a mortar and sieved to obtain a 40 to 60 mesh size powder, and 24 mg of the powder was filled in a catalyst tube. Subsequently, while helium was supplied at 14 mL/min in the manufacturing apparatus, the raw material supply part and the continuous reactor were heated at 200° C. and 500° C., respectively, for 1 hour, and then the temperature of the continuous reactor was lowered to 300° C. and stabilized while the raw material supply part was kept at 200° C. The reaction mixture recovery part was cooled with liquid nitrogen. Subsequently, the molar ratio of ethanol to 2,5-dimethylfuran was set within the range of 1.0 to 30.5, and 6 μL of the mixed solution was injected from the raw material supply port with a microsyringe. The reaction mixture obtained in the reaction mixture recovery part 30 minutes after injection was analyzed by GC, and the yield of each component was calculated to obtain the results shown in Table 2.

From the results of Examples 8 to 12 shown in Table 2, it was found that the yield of para-xylene and the aromatic hydrocarbons tended to be improved as the molar ratio of ethanol to the furan derivative was larger.

TABLE 2

| | Example 8 | Example 9 | Example 10 | Example 11 | Example 12 |
|---|---|---|---|---|---|
| EtOH/DMF ratio | 1.0 | 5.0 | 10.2 | 15.3 | 30.5 |
| Total aromatic | 18.2 | 50.7 | 62.7 | 86.5 | 73.1 |
| hydrocarbons (%/DMF) | | | | | |
| Total xylenes (%/DMF) | 7.3 | 23.3 | 29.0 | 30.8 | 36.3 |
| PX (%/DMF) | 6.8 | 20.9 | 27.0 | 28.7 | 32.4 |
| 2,5-Hexanedione (%/DMF) | 1.2 | 1.1 | 1.2 | 0.8 | 0.7 |

### Examples 13 to 15

ZSM-5 (HSZ/840HOD1A manufactured by Tosoh Corporation) was pulverized in a mortar and sieved to obtain a 40 to 60 mesh size powder, and 24 mg of the powder was filled in a catalyst tube. Subsequently, while helium was supplied at 14 mL/min in the manufacturing apparatus, the raw material supply part and the continuous reactor were heated at 200° C. and 500° C., respectively, for 1 hour, and then the temperature of the continuous reactor was set to 200 to 400° C. and stabilized while the raw material supply part was kept at 200° C. The reaction mixture recovery part was cooled with liquid nitrogen. Subsequently, 6 μL of a mixed solution in which the molar ratio of ethanol to 2,5-dimethylfuran was 30.5 was injected from the raw material supply port with a microsyringe. The reaction mixture obtained in the reaction mixture recovery part 30 minutes after injection was analyzed by GC, and the yield of each component was calculated to obtain the results shown in Table 3.

From the results of Examples 13 to 15 shown in Table 3, it can be confirmed that aromatic hydrocarbons are obtained in the range of 200 to 400° C., and the yield is improved as the temperature is higher. On the other hand, it was found that a lower temperature tends to be more advantageous to the para-xylene selectivity, and it is good to set the conditions considering the balance with the reactivity.

TABLE 3

| | Example 13 | Example 14 | Example 12 | Example 15 |
|---|---|---|---|---|
| Temperature (° C.) | 200 | 250 | 300 | 400 |
| Total aromatic | 5.0 | 33.2 | 73.1 | 100 |
| hydrocarbons (%/DMF) | | | | |
| Total xylenes (%/DMF) | 0.0 | 19.1 | 36.3 | 44.1 |
| PX (%/DMF) | 0.0 | 17.0 | 32.4 | 38.3 |
| 2,5-Hexanedione (%/DMF) | 0.0 | 0.0 | 0.7 | 0.8 |

### Example 16

ZSM-5 (HSZ/840HOD1A manufactured by Tosoh Corporation) was pulverized in a mortar and sieved to obtain a 40 to 60 mesh size powder, and 24 mg of the powder was filled in a catalyst tube. Subsequently, while helium was supplied at 14 mL/min in the manufacturing apparatus, the raw material supply part and the continuous reactor were heated at 200° C. and 500° C., respectively, for 1 hour, and then the temperature of the continuous reactor was lowered to 300° C. and stabilized while the raw material supply part was kept at 200° C. The reaction mixture recovery part was cooled with liquid nitrogen. Subsequently, 1.5 μL of a mixed solution in which the molar ratio of ethanol to 2,5-dimethylfuran was 2.0 was injected from the raw material supply port with a microsyringe. The reaction mixture obtained in the reaction mixture recovery part 30 minutes after injection was analyzed by GC, and the yield of each component was calculated to obtain the results shown in Table 4.

### Example 17

The same procedures as in Example 16 were carried out except that ethanol in the raw material compounds was changed to ethylene gas (415 μL), which was injected simultaneously with 2,5-dimethylfuran (1.0 μL).

From the results of Examples 16 and 17, it was found that ethanol and ethylene can be used in the same manner.

TABLE 4

| | Example 16 | Example 17 |
|---|---|---|
| EtOH/DMF molar ratio | 2.0 | — |
| Ethylene/DMF molar ratio | — | 2.0 |
| Total aromatic | 35.1 | 34.6 |
| hydrocarbons (%/DMF) | | |
| Total xylenes (%/DMF) | 13.2 | 15.0 |
| PX (%/DMF) | 12.1 | 13.5 |
| 2,5-Hexanedione (%/DMF) | 0.4 | 0.3 |

### Example 18

ZSM-5 (HSZ/840HOD1A manufactured by Tosoh Corporation) was pulverized in a mortar and sieved to obtain a 40 to 60 mesh size powder, and 24 mg of the powder was filled in a catalyst tube. Subsequently, while helium was supplied at 14 mL/min in the manufacturing apparatus, the raw material supply part and the continuous reactor were heated at 200° C. and 500° C., respectively, for 1 hour, and then the temperature of the continuous reactor was lowered to 300° C. and stabilized while the raw material supply part was kept at 200° C. The reaction mixture recovery part was cooled with liquid nitrogen. Subsequently, 1.0 μL of a mixed solution in which the molar ratio of ethanol to 2,5-dimethylfuran was 30.5 was injected 6 times at intervals of 1 minute from the raw material supply port with a microsyringe. The reaction mixture obtained in the reaction mixture recovery part 30 minutes after the final injection was analyzed by GC, and the yield of each component was calculated to obtain the results shown in Table 5.

### Examples 19 to 21

ZSM-5 (HSZ/840HOD1A manufactured by Tosoh Corporation) was pulverized in a mortar and sieved to obtain a 40 to 60 mesh size powder, and 24 mg of the powder was filled in a catalyst tube. Subsequently, while helium was supplied at 14 mL/min in the manufacturing apparatus, the raw material supply part and the continuous reactor were heated at 200° C. and 500° C., respectively, for 1 hour, and then the temperature of the continuous reactor was lowered to 300° C. and stabilized while the raw material supply part was kept at 200° C. The reaction mixture recovery part was cooled with liquid nitrogen. Subsequently, a mixed solution in which the molar ratio of ethanol to 2,5-dimethylfuran was 30.5 was injected from the raw material supply port at a flow rate set in the range of 1.0 to 4.0 μL/min with a micro-feeder. The reaction mixture obtained in the reaction mixture recovery part over 6 minutes from 30 minutes after the start of injection was analyzed by GC, and the yield of each component was calculated to obtain the results shown in Table 5.

TABLE 5

| | Example 12 | Example 18 | Example 19 | Example 20 | Example 21 |
|---|---|---|---|---|---|
| Supply method | Supply at a | Repeated supply | Continuous | Continuous | Continuous |
| | time | at a time | supply | supply | supply |
| Supply amount (μL) | 6 | 1 × 6 times | 1/min | 2/min | 4/min |
| Total aromatic | 73.1 | 100 | 49.6 | 59.6 | 11.3 |
| hydrocarbons (%/DMF) | | | | | |
| Total xylenes (%/DMF) | 36.3 | 52.0 | 22.5 | 16.6 | 2.7 |
| PX (%/DMF) | 32.4 | 44.3 | 19.1 | 14.8 | 2.1 |
| 2,5-Hexanedione (%/DMF) | 0.7 | 1.2 | 1.1 | 1.1 | 0.2 |

### Comparative Example 1

An example in which the reaction was carried out in a batch method in an autoclave is shown with reference to a non-patent document (Angew. Chem. Int. Ed. 2016, 55, 13061-13066).

In a stainless steel 100 mL autoclave, a 40 to 60 mesh size powder (1.05 g) obtained by pulverizing 2,5-dimethylfuran (18.0 mL), ethanol (10.0 mL), and ZSM-5 (HSZ/840HOD1A manufactured by Tosoh Corporation) in a mortar and sieving the mixture was charged. The inside of the autoclave was replaced with nitrogen, and then the autoclave was sealed. Here, the molar ratio of ethanol to 2,5-dimethylfuran was 1.0. Subsequently, while stirring the mixture in the autoclave, the internal temperature was raised to 300° C., held for 6 hours, and then cooled to room temperature. The catalyst was removed from the reaction mixture, and the resulting reaction solution was analyzed by GC. As a result, as shown in Table 6, the production rate of an aromatic hydrocarbon to 2,5-dimethylfuran in the raw material was 0.5%, and the reaction hardly proceeded.

### Comparative Example 2

The same procedures as in Comparative Example 1 were carried out except that the volume of 2,5-dimethylfuran was changed to 3.4 mL, the volume of ethanol was changed to 27.9 mL, and the molar ratio of ethanol to 2,5-dimethylfuran was changed to 15.2. The molar ratio of the raw material compounds was obtained by applying the molar ratio of Example 11. As a result of analyzing the obtained reaction liquid by GC, as shown in Table 6, the production rate of an aromatic hydrocarbon to 2,5-dimethylfuran in the raw material was 5.7%, which was insufficient as compared with the non-patent document. In addition, para-xylene was 0.3%, and only a trace amount of para-xylene was obtained.

Comparison of the results of Comparative Example 1 also showed that aromatic hydrocarbons were efficiently obtained in an extremely short time by the manufacturing method of the present invention.

TABLE 6

| | Comparative | Comparative |
|---|---|---|
| | Example 1 | Example 2 |
| EtOH/DMF ratio | 1.0 | 15.2 |
| Total aromatic | 0.5 | 5.7 |
| hydrocarbons (%/DMF) | | |
| Total xylenes (%/DMF) | — | 2.6 |
| PX (%/DMF) | — | 0.3 |

### INDUSTRIAL APPLICABILITY

According to the present invention, an aromatic hydrocarbon useful as a polymer raw material or the like can be efficiently obtained with high purity. Furthermore, when biomass-derived 2,5-dimethylfurfural and biomass-derived ethanol are used, an 100%-biomass-derived aromatic hydrocarbon is obtained. A polyester completely derived from biomass can be obtained by using such para-xylene completely derived from biomass in combination with a glycol derived from biomass.

### DESCRIPTION OF REFERENCE SIGNS

1: Gas flow rate controller
2: Raw material vaporizer
3: Raw material supply part
4: Continuous reactor
5: Reaction mixture recovery part
6: Inert gas
7: Raw material compound
8: Reaction mixture

1. A method for manufacturing an aromatic hydrocarbon, comprising contacting ethanol and/or ethylene, and a furan derivative with a catalyst in a continuous reactor.

2. The method for manufacturing an aromatic hydrocarbon according to claim 1,
wherein ethanol is brought into contact with the catalyst in a continuous reactor to convert at least a part of the ethanol into ethylene, and
the ethylene and the furan derivative are brought into contact with the catalyst in a continuous reactor.

3. The method for manufacturing an aromatic hydrocarbon according to claim 2, wherein the conversion of ethanol to ethylene and the contact of ethylene and the furan derivative with the catalyst are performed in the same continuous reactor.

4. The method for manufacturing an aromatic hydrocarbon according to claim 1, wherein ethanol and/or ethylene and the furan derivative are brought into contact with the catalyst in a gaseous state.

5. The method for manufacturing an aromatic hydrocarbon according to claim 1, wherein a molar ratio of ethanol and/or ethylene (in a case where both ethanol and ethylene are contained, a total of ethanol and ethylene) to the furan derivative to be brought into contact with the catalyst is 1.0 or more and 50.0 or less.

6. The method for manufacturing an aromatic hydrocarbon according to claim 1, wherein a pressure in the continuous reactor is 1.0 MPa or less.

7. The method for manufacturing an aromatic hydrocarbon according to claim 1, wherein the at least one catalyst contains a solid acid.

8. The method for manufacturing an aromatic hydrocarbon according to claim 7, wherein the solid acid is at least one selected from the group consisting of zeolite, alumina, and a heteropolyacid.

9. The method for manufacturing an aromatic hydrocarbon according to claim 1, wherein the furan derivative is derived from biomass.

10. The method for manufacturing an aromatic hydrocarbon according to claim 1, wherein ethanol and/or ethylene is derived from biomass.

11. An aromatic hydrocarbon obtained by the method for manufacturing an aromatic hydrocarbon according to claim 1.

12. A method for manufacturing a polymer, comprising:
manufacturing the aromatic hydrocarbon by the method for manufacturing an aromatic hydrocarbon according to claim 1; and
manufacturing a polymer using the resulting aromatic hydrocarbon as a raw material.

13. An apparatus for manufacturing an aromatic hydrocarbon, comprising:
a raw material supply part;
a flow type continuous reactor filled with a catalyst; and
a reaction mixture recovery part,
wherein the raw material supply part includes supply means for continuously supplying a raw material compound containing ethanol and/or ethylene, and a furan derivative to the continuous reactor, and
the reaction mixture recovery part includes discharge means for continuously extracting a reaction mixture having been in contact with the catalyst from the continuous reactor.

14. The apparatus for manufacturing an aromatic hydrocarbon according to claim 13, wherein the raw material supply part further includes vaporization means for vaporizing ethanol and the furan derivative.

15. The apparatus for manufacturing an aromatic hydrocarbon according to claim 13, wherein the reaction mixture recovery part further includes condensation means for condensing at least a part of the extracted reaction mixture.

16. The apparatus for manufacturing an aromatic hydrocarbon according to claim 13, further including pressure control means capable of controlling an internal pressure of the continuous reactor within a range of 1.0 MPa or less.
